(19) United States
(12) Patent Application Publication
(10) Pub. No.: US 2024/0000552 A1
(43) Pub. Date: Jan. 4, 2024

(54) OCT DEVICE, METHOD FOR CONTROLLING SAME, AND OCT DEVICE CONTROLLING PROGRAM

(71) Applicant: THE YOSHIDA DENTAL MFG. CO., LTD., Tokyo (JP)

(72) Inventor: Takanobu SAGAWA, Tokyo (JP)

(21) Appl. No.: 18/253,413

(22) PCT Filed: Mar. 8, 2022
(86) PCT No.: PCT/JP2022/009932

§ 371 Date: May 18, 2023

(30) Foreign Application Priority Data
Mar. 15, 2021 (JP) 2021-041221

(51) Int. Cl. A61C 9/00; G01B 9/02091

(52) U.S. Cl. CPC A61C 9/0053; G01B 9/02091

(57) ABSTRACT

OCT device (1) includes: a signal processor (110) obtaining preview image data corresponding to a volume image with predetermined graininess by a single image capture by measuring subject information in a direction along the optical axis of the laser beam at a preset point interval for two-dimensional scanning; a memory (120); and an instruction receiving means (130). The signal processor (120) obtains pieces of the preview image data by a plurality of image captures with an origin of the two-dimensional scanning shifted by a small interval smaller than the point interval so as to divide the point interval each time a piece of the preview image data is acquired, and generates measurement-image-capture image data by reconstructing pieces of the preview image data obtained by a plurality of image captures and stored in the memory (120) up to a point when an instruction was received.

### TECHNICAL FIELD

The present invention relates to an OCT apparatus, a method for controlling the same, and an OCT apparatus controlling program, and relates particularly to a dental OCT apparatus, a method for controlling the same, and an OCT apparatus controlling program.

### BACKGROUND ART

Heretofore, an optical coherence tomography apparatus (OCT apparatus) has been known which measures internal information of a subject by optical coherence by irradiating the subject with a laser beam (see Patent Literature 1). The OCT apparatus disclosed in Patent Literature 1 is equipped with two image capture modes—a mode actuated in response to a measurement instruction (hereinafter referred to as “measurement-mode image capture”) and a mode actuated in response to a preview instruction (hereinafter referred to as “preview-mode image capture”).

In the measurement-mode image capture, the OCT apparatus obtains an optical coherence tomography image (OCT image) of a subject with a predetermined resolution by causing a scanning mechanism to perform a scan at predetermined intervals, and a display device displays the OCT image as a still image. In the measurement-mode image capture, subject image data is expected to be stored. When the subject is a tooth, the operator brings a nozzle tip of a probe of the OCT apparatus into contact with the tooth, and asks the patient not to move for several seconds until the measurement image capture ends.

In the preview-mode image capture, on the other hand, subject image data is not expected to be stored. Thus, the OCT apparatus obtains an image with a lower resolution by causing the scanning mechanism to perform a scan at larger intervals. In the preview-mode image capture, the OCT apparatus obtains images with the lower resolution in sequence, and the display device in turn quickly displays the subject's tomographic image, en-face image, or three-dimensional image as a real-time moving image. For this reason, the preview-mode image capture is sometimes used before the measurement-mode image capture to determine the position of the image capture spot for obtaining an image to be stored. Note that an en-face image is a two-dimensional image obtained by combining pieces of hight-direction data in a subject's three-dimensional image. Unlike simple surface images and elevational images, en-face images are generated by utilizing not only information of a subject's outer surface but also internal information.

### CITATION LIST

### Non-Patent Literature

Patent Literature 1: JP5827024

### SUMMARY OF INVENTION

### Technical Problem

The conventional OCT apparatus can obtain a good image appropriate for storage by the measurement-mode image capture, but may also obtain an image not appropriate for storage. For example, if the subject moves or the image capture probe is moved during the image capture, it distorts the image, so that an image not appropriate for storage is obtained. Also, if, for example, blood, saliva, or the like oozes over the subject during the image capture, the laser beam gets absorbed or reflected at the subject's surface, so that an image not appropriate for storage is obtained. When an image not appropriate for storage is obtained as above, it is necessary to redo the image capture. Thus, there was room for improvement in the OCT apparatus.

The present invention has been made in view of the above circumstance, and an object thereof is to provide an OCT apparatus, a method for controlling the same, and an OCT apparatus controlling program which are capable of reducing the number of times to redo an image capture to obtain a good image appropriate for storage.

### Solution to Problem

An OCT apparatus according to the present invention to solve the above object is an OCT apparatus for measuring internal information of a subject by optical coherence by irradiating the subject with a laser beam from a probe including a two-dimensional scanning mechanism that two-dimensionally scans a laser beam, the OCT apparatus including: a signal processor that obtains preview image data corresponding to a volume image with predetermined graininess by a single image capture, the volume image being sectional images of cross sections along an optical axis of a laser beam stacked in a direction perpendicular to the cross sections by measuring subject information in a direction along the optical axis of the laser beam at a preset point interval for two-dimensional scanning; a memory that stores pieces of the preview image data obtained by a plurality of image captures; and an instruction receiver that receives an instruction to generate measurement-image-capture image data corresponding to a volume image of the subject with finer graininess than the predetermined graininess, in which the signal processor obtains pieces of the preview image data by a plurality of image captures with an origin of the two-dimensional scanning shifted by a small interval smaller than the point interval so as to divide the point interval each time the signal processor obtains a piece of the preview image data, and generates the measurement-image-capture image data by reconstructing pieces of the preview image data obtained by a plurality of image captures and stored in the memory up to a point when the instruction was received.

A method for controlling an OCT apparatus according to the present invention to solve the above object is a method for controlling an OCT apparatus for measuring internal information of a subject by optical coherence by irradiating the subject with a laser beam from a probe including a two-dimensional scanning mechanism that two-dimensionally scans a laser beam, the method including: a step of obtaining preview image data corresponding to a volume image with predetermined graininess by a single image capture, the volume image being sectional images of cross sections along an optical axis of a laser beam stacked in a direction perpendicular to the cross sections by measuring subject information in a direction along the optical axis of the laser beam at a preset point interval for two-dimensional scanning; a step of shifting an origin of the two-dimensional scanning by a small interval smaller than the point interval so as to divide the point interval each time a piece of the preview image data is obtained; a step of storing pieces of the preview image data obtained by a plurality of image captures in a memory; a step of receiving an instruction to generate measurement-image-capture image data corresponding to a volume image of the subject with finer graininess than the predetermined graininess; and a step of generating the measurement-image-capture image data by reconstructing pieces of the preview image data obtained by a plurality of image captures and stored in the memory up to a point when the instruction was received.

Note that the present invention can also be implemented with a program for causing a computer to function as a control device for the OCT apparatus described above.

### Advantageous Effect of Invention

According to the present invention, it is not necessary to perform a preview-mode image capture and then perform a measurement-mode image capture. This reduces the number of images not appropriate for storage and reduces the number of times to redo an image capture.

### BRIEF DESCRIPTION OF DRAWINGS

FIG. 1 is a configuration diagram schematically showing an OCT apparatus according to a first embodiment of the present invention.

FIG. 2 is a block diagram schematically showing a configuration of a control unit in FIG. 1.

FIG. 3 is a flowchart showing a flow of image generation-display processing by the OCT apparatus according to the first embodiment of the present invention.

FIGS. 4A and 4B are explanatory diagrams of a process of generating measurement-image-capture image data according to the first embodiment of the present invention, FIG. 4A showing an A-sectional image, FIG. 4B showing a volume image.

FIGS. 5A and 5B are explanatory diagrams of a process of generating preview image data according to the first embodiment of the present invention, FIG. 5A showing an A-sectional image, FIG. 5B showing a volume image.

FIG. 6 is a schematic diagram showing a flow of an image capture process until a measurement-image-capture image is displayed according to the conventional art.

FIG. 7 is a schematic diagram showing a flow of an image capture process until a measurement-image-capture image is displayed according to the first embodiment of the present invention.

FIG. 8 is a schematic diagram showing an advantageous effect of the OCT apparatus according to the first embodiment of the present invention.

FIG. 9 is a block diagram schematically showing a configuration of a control unit of an OCT apparatus according to a second embodiment of the present invention.

FIG. 10 is a schematic diagram showing a flow of an image capture process until a measurement-image-capture image is displayed according to the second embodiment of the present invention.

FIG. 11 is a block diagram schematically showing a configuration of a control unit of an OCT apparatus according to a third embodiment of the present invention.

FIG. 12 is a block diagram schematically showing a configuration of a control unit of an OCT apparatus according to a fourth embodiment of the present invention.

FIG. 13 is a block diagram schematically showing a configuration of a control unit of an OCT apparatus according to a fifth embodiment of the present invention.

FIG. 14 is an explanatory diagram of a shake detection means in FIG. 13.

FIG. 15 is a block diagram schematically showing a configuration of a control unit of an OCT apparatus according to a sixth embodiment of the present invention.

FIG. 16 is a display example of a screen on the OCT apparatus in FIG. 15.

### DESCRIPTION OF EMBODIMENTS

Embodiments of an OCT apparatus according to the present invention will be described in detail with reference to the drawings. Note that the sizes of the members shown in some drawings as well as their positional relationships and the like may be exaggerated for clear explanation.

### First Embodiment

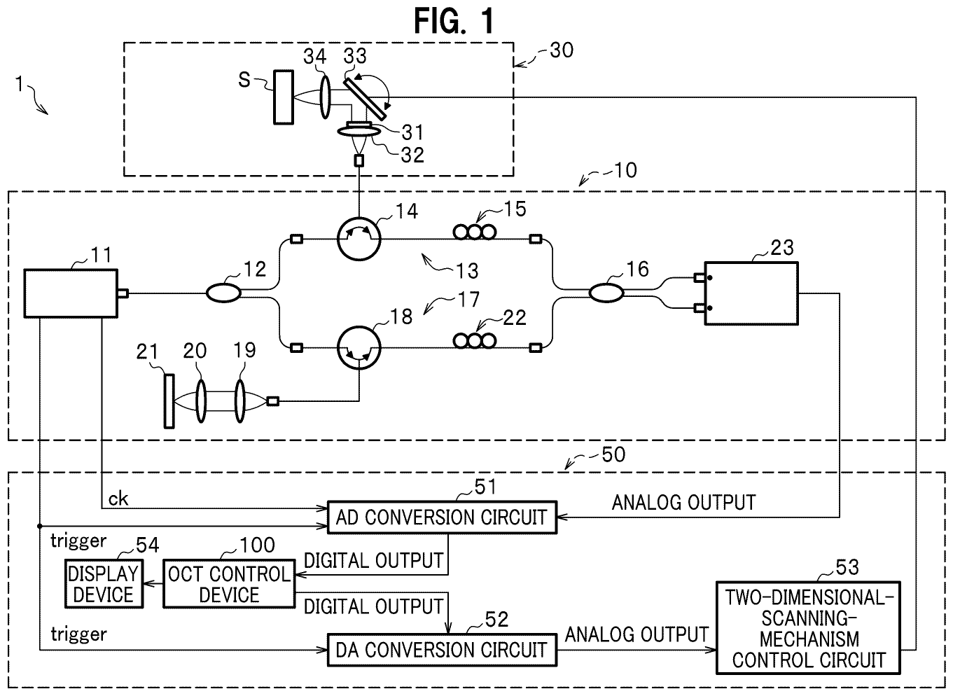

As shown in FIG. 1, an OCT apparatus 1 according to a first embodiment mainly includes an optical unit 10, a probe 30, and a control unit 50, and measures internal information of a subject S by optical coherence by irradiating the subject S with a laser beam.

The optical unit 10 includes a light source, optical systems, and detectors supporting general methods of optical coherence tomography. The optical unit 10 includes a light source 11 that periodically irradiates the subject S with a laser beam, a detector 23 that detects the internal information of the subject S, optical fibers and various optical parts provided in optical paths between the light source 11 and the detector 23, and so on. An SS-OCT (Swept Source Optical Coherence Tomography)-type laser beam output device, for example, is usable as the light source 11. The subject S is a tooth, for example.

The optical unit 10 will now be described briefly. A light beam emitted from the light source 11 is slit into a measurement light beam and a reference light beam by a coupler 12 serving as a light splitter. The measurement light beam enters the probe 30 from a circulator 14 of a sample arm 13. When a shutter 31 of the probe 30 is open, this measurement light beam passes a collimator lens 32 and a two-dimensional scanning mechanism 33 and is focused at the subject S by a condensing lens 34. After being scattered and reflected there, the measurement light beam passes the condensing lens 34, the two-dimensional scanning mechanism 33, and the collimator lens 32 again and returns to the circulator 14 of the sample arm 13. The returned measurement light beam enters the detector 23 through a coupler 16.

On the other hand, the reference light beam separated by the coupler 12 passes a collimator lens 19 from a circulator 18 of a reference arm 17 and focused at a reference minor 21 by a condensing lens 20. After being reflected there, the reference light beam passes the condensing lens 20 and the collimator lens 19 again and returns to the circulator 18. The returned reference light beam enters the detector 23 through the coupler 16. This means that the coupler 16 combines the measurement light beam scatted and reflected at and returned from the subject S and the reference light beam reflected at the reference mirror 21. The detector 23 can therefore detect the light beams interfering with each other as a result of being combined (coherent light beams) as the internal information of the subject S. Note that a polarization controller 15 of the sample arm 13 and a polarization controller 22 of the reference arm 17 are each installed in order to reduce the polarization occurring inside the OCT apparatus 1, which includes the probe 30, back to lower polarization.

The probe 30 includes the two-dimensional scanning mechanism 33, which scans a laser beam two-dimensionally, and guides the laser beam from the optical unit 10 to the subject S and also guides the light beam reflected on the subject S to the optical unit 10. In the present embodiment, the two-dimensional scanning mechanism 33 includes two galvanometer minors whose rotation axes are perpendicular to each other, motors for the galvanometer minors, and so on.

The control unit 50 includes an AD conversion circuit 51, a DA conversion circuit 52, a two-dimensional-scanning-mechanism control circuit 53, a display device 54, and an OCT control device 100.

The AD conversion circuit 51 converts an analog output signal from the detector 23 into a digital signal. In the present embodiment, the AD conversion circuit 51 starts acquiring signals in synchronization with a trigger output from a laser output device which is the light source 11 and also acquires the analog output signal from the detector 23 and converts it into a digital signal with the timing of a clock signal ck output from the same laser output device. This digital signal is input to the OCT control device 100.

The DA conversion circuit 52 converts a digital output signal from the OCT control device 100 into an analog signal. In the present embodiment, the DA conversion circuit 52 converts the digital signal from the OCT control device 100 into an analog signal in synchronization with the trigger output from the light source 11. This analog signal is inputted to the two-dimensional-scanning-mechanism control circuit 53.

The two-dimensional-scanning-mechanism control circuit 53 is a driver that controls the two-dimensional scanning mechanism 33 in the probe 30. The two-dimensional-scanning-mechanism control circuit 53 outputs motor driving signals that drive or stop the motors for the galvanometer mirrors in synchronization with the output periodic intervals of the laser beam emitted from the light source 11 based on the analog output signal from the OCT control device 100. The two-dimensional-scanning-mechanism control circuit 53 performs a process of turning the rotation axis of one galvanometer minor to change the angle of its minor surface and a process of turning the rotation axis of the other galvanometer minor to change the angle of its minor surface at different timings.

The display device 54 displays an optical coherence tomography image (hereinafter referred as “OCT image”) generated by the OCT control device 100. The display device 54 is a liquid crystal display (LCD) or the like, for example.

The OCT control device (OCT apparatus control device) 100 is a device that controls the OCT apparatus 1, and captures an image by controlling the two-dimensional scanning mechanism 33 in synchronization with a laser beam emitted from the light source 11, and also performs control for generating an OCT image of the subject S from data obtained by converting the detection signal from the detector 23. The OCT image and the like can be generated by a publicly known method for generating an optical coherence tomography image and the like. Note that the OCT image and the like can be generated using the method disclosed in Patent Literature 1, for example.

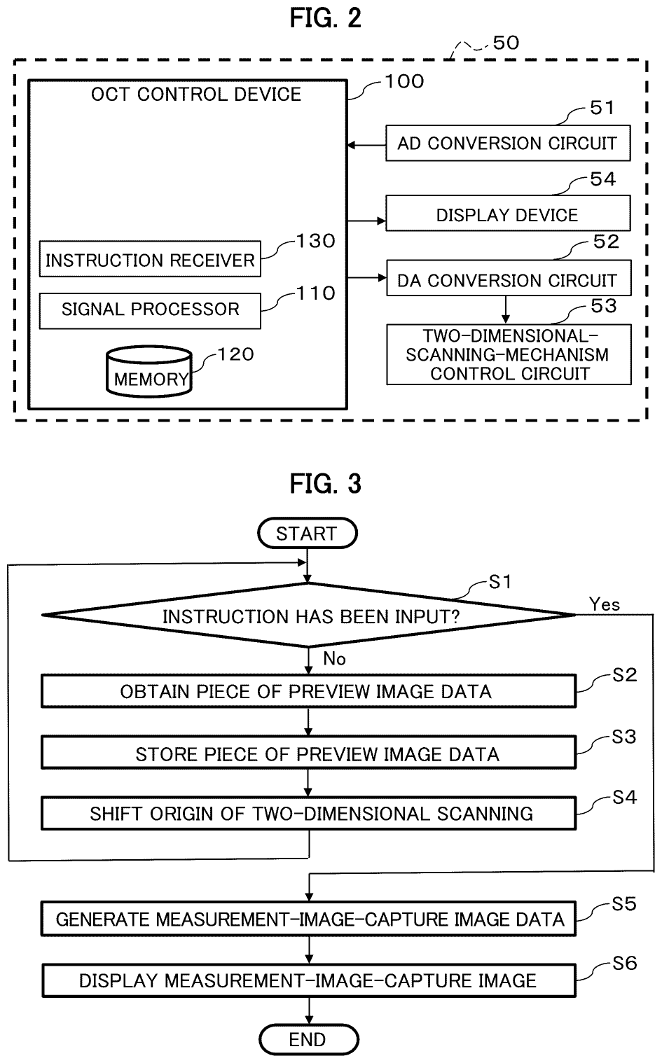

The configuration of the OCT apparatus 1 will now be described in further detail focusing on the OCT control device 100 with reference to FIG. 2. The OCT control device 100 includes a signal processor 110, a memory 120, and an instruction receiver 130.

The signal processor 110 obtains a piece of preview image data representing a volume image with predetermined graininess by a single image capture. This volume image with the predetermined graininess is sectional images of cross sections along the optical axis of a laser beam stacked in the direction perpendicular to the cross sections by measuring subject information in a direction along the optical axis of the laser beam at preset point intervals for two-dimensional scanning. This function of the signal processor 110 is similar to the function of the preview-mode image capture by the conventional OCT apparatus.

The memory 120 stores pieces of preview image data obtained by a plurality of image captures. The memory 120 sequentially stores pieces of preview image data obtained by a plurality of image captures and, when the storage capacity is exceeded, the memory 120 sequentially erases the pieces of preview image data in chronological order from the oldest.

The instruction receiver 130 receives an instruction to generate finer image data than preview image data. Here, the finer image data is measurement-image-capture image data corresponding to a volume image of the subject S with finer graininess than the predetermined graininess. Meanwhile, the OCT apparatus 1 in the present embodiment does not perform measurement-mode image capture. Nonetheless, the finer image data is data appropriate for storage like data obtained by the measurement-mode image capture by the conventional OCT apparatus, and will therefore be referred to as “measurement-image-capture image data”.

The instruction receiver 130 determines that an instruction to generate finer image data than preview image data is received when an image capture button not shown is clicked.

The signal processor 110 obtains pieces of preview image data by a plurality of image captures with the origin of two-dimensional scanning shifted by a small interval smaller than a point interval so as to divide the point interval each time the signal processor 110 obtains a piece of preview image data. In the following, the origin of two-dimensional scanning will also be referred to as “scan origin”. The signal processor 110 generates measurement-image-capture image data by reconstructing the pieces of preview image data obtained by a plurality of (e.g., nine) image captures and stored in the memory 120 up to the point when the instruction receiver 130 received the instruction. As a result, the display device 54 displays a measurement-image-capture image (still image).

The method for shifting the scan origin can be a mechanical method or a software method, for example. In the present embodiment, a mechanical method is employed in which, each time the signal processor 110 obtains a piece of preview image data, the signal processor 110 shifts the scan origin (the origin of two-dimensional scanning) by outputting a signal for shifting the origin of a scanning start position at the two-dimensional scanning mechanism 33 to the two-dimensional-scanning-mechanism control circuit 53, which controls the two-dimensional scanning mechanism 33.

The OCT control device 100 is, for example, a computer including a CPU (Central Processing Unit), a GPU (Graphics Processing Unit), a RAM (Random Access Memory), a ROM (Read Only Memory), a hard disk drive, and an input-output interface.

Next, a control method by the OCT apparatus 1 will be described focusing on the OCT control device 100. The control method by the OCT apparatus 1 includes a preview image data obtaining step, an origin moving step, a storing step, an instruction receiving step, and a measurement-image-capture image data generating step.

The preview image data generating step is a step of obtaining a piece of preview image data corresponding to a volume image with the predetermined graininess by a single image capture, the volume image being sectional images of cross sections along the optical axis of a laser beam stacked in the direction perpendicular to the cross sections by measuring subject information in a direction along the optical axis of the laser beam at preset point intervals for two-dimensional scanning.

The origin moving step is a step of shifting the origin of two-dimensional scanning by a small interval smaller than a point interval so as to divide the point interval each time a piece of preview image data is obtained.

The storing step is a step of storing pieces of preview image data obtained by a plurality of image captures in the memory 120.

The instruction receiving step is a step of receiving an instruction to generate measurement-image-capture image data corresponding to a volume image of the subject S with finer graininess than the predetermined graininess.

The measurement-image-capture image data generating step is a step of generating the measurement-image-capture image data by reconstructing the pieces of preview image data obtained by a plurality of image captures and stored in the memory 120 up to the point when the instruction was received.

Next, a flow of image generation-display processing by the OCT apparatus will be described with reference to FIG. 3 (see FIG. 2 as appropriate). First, the instruction receiver 130 determines whether an instruction has been input (step S1). Specifically, the instruction receiver 130 determines whether the image capture button has been clicked. If no instruction has been input (step S1: No), the signal processor 110 obtains a piece of preview image data corresponding to a single image capture (step S2), and stores it in the memory 120 (step S3). The OCT apparatus 1 then performs a process of shifting the scan origin (origin of two-dimensional scanning) (step S4) and returns to step S1. On the other hand, if the image capture button is clicked and an instruction is input in step S1 mentioned above (step S1: Yes), the signal processor 110 generates measurement-image-capture image data by reconstructing the pieces of preview image data obtained by a plurality of image captures and stored in the memory 120 up to that point (step S5), and the display device 54 displays a measurement-image-capture image (step S6).

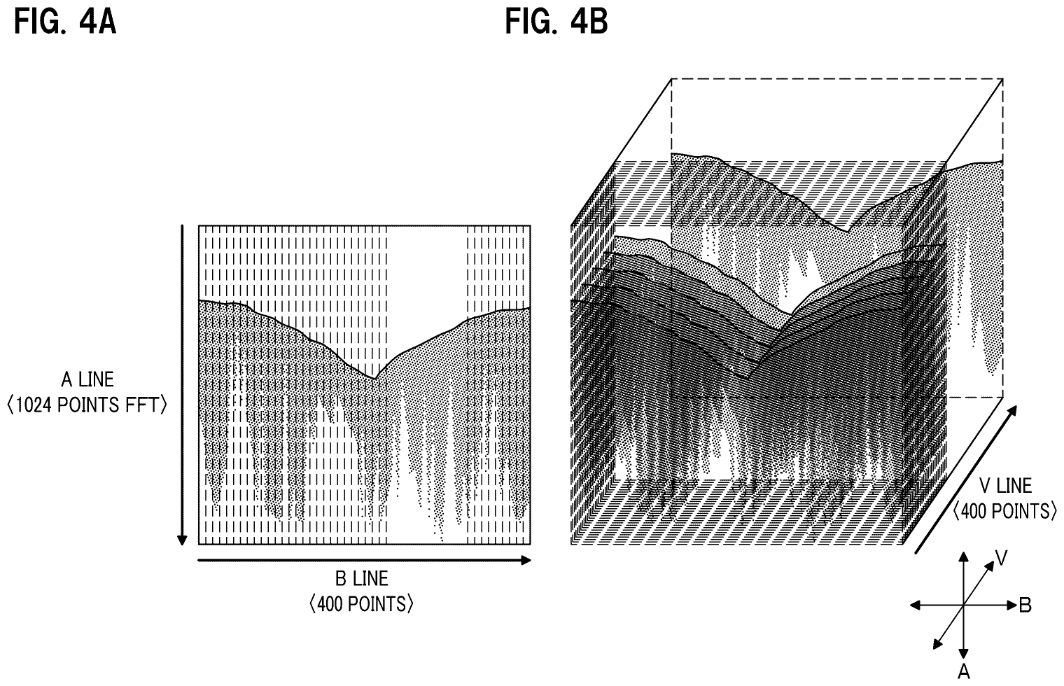

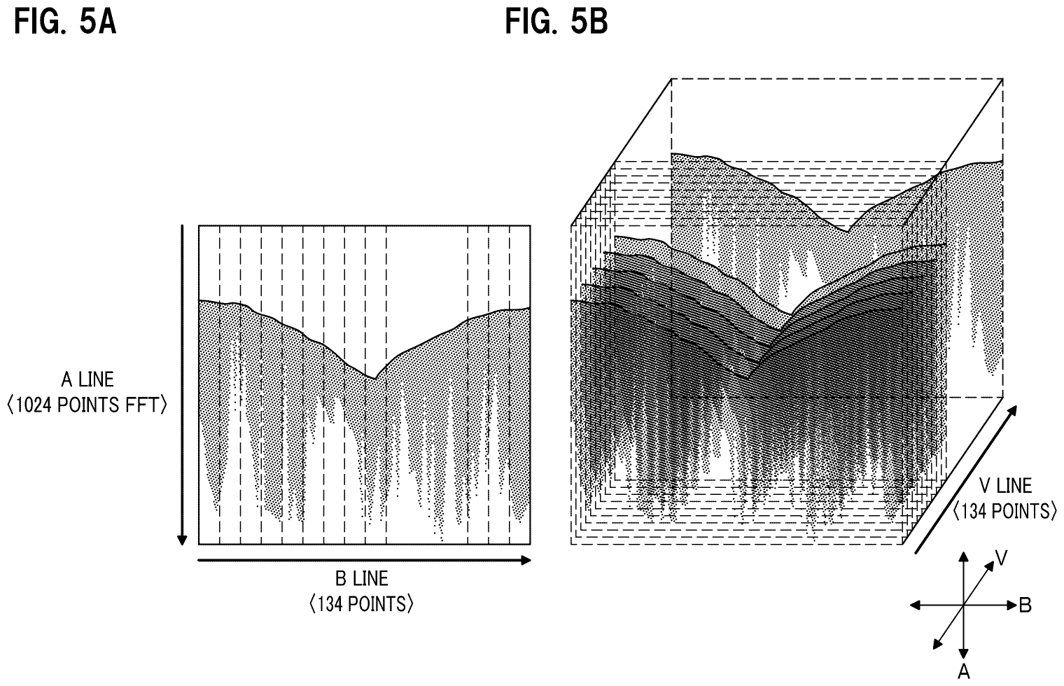

Next, a specific example of a piece of preview image data and measurement-image-capture image data generated by the OCT apparatus will be described with reference to FIG. 5 (see FIGS. 4 and 2 as appropriate). The A line shown in FIG. 5A indicates the direction of emission of a laser beam along the optical axis of the condensing lens 34 (A axis) in the probe 30. A piece of data in the A-axis direction (hereinafter referred to as “A-line data”) is data indicating tomographic information (internal information) of the subject S in the hight direction from its surface. The B line shown in FIG. 5A extends along the optical axis of the collimator lens 32 (B-axis direction) in the probe 30. The B line is set in the width direction of the subject S by reciprocating movement of one galvanometer minor. In the following, the tomographic image schematically shown in FIG. 5A will be referred to as “A-sectional image”. The V line shown in FIG. 5B extends along the direction perpendicular to each of the A and B axes. The V line is set in the depth direction of the subject S by reciprocating movement of the other galvanometer mirror. FIG. 5B schematically shows that a three-dimensional image can be formed by stacking A-sectional images in the direction perpendicular to their planes (V-axis direction).

Incidentally, in an example disclosed in Patent Literature 1 (comparative example), the number of points on the B line and the number of points on the V line in one measurement-mode image capture (one volume) are each 400 points (see FIG. 4).

### Comparative Example: Conditions for Conventional Measurement-Mode Image Capture

A line: 1024 points

B line: 400 points

V line: 400 points

FIG. 5A and FIG. 5B are schematic diagrams of a case where the number of points on the B line and the number of points on the V line in one preview-mode image capture (one volume) in the present embodiment are each set at 134 points, which is ⅓ of 400 points, based on back calculation from the conditions in Comparative Example above.

### Example: Conditions for Preview-mode Image Capture

A line: 1024 points

B line: 134 points (approximately ⅓ of the points in the measurement-mode image capture)

V line: 134 points (approximately ⅓ of the points in the measurement-mode image capture)

In other words, in Example, a point interval on the B line in a preview-mode image capture is approximately three times longer than a point interval on the B line in a measurement-mode image capture. Also, in Example, a point interval on the V line in a preview-mode image capture is approximately three times longer than a point interval on the V line in a measurement-mode image capture.

In this case, in the present embodiment, the OCT apparatus 1 performs a scan with the scan origin shifted by ⅓ of a point interval on the B line or by ⅓ of a point interval on the V line so as to divide the point interval after each one preview-mode image capture (one volume). In the following, this operation will be referred to as “interlace scanning”. The number of points set in one scan (one volume) in the interlace scanning is the same as the number of points set in the conventional preview-mode image capture, and an image displayed as a result of one scan in the interlace scanning is similar to an image displayed as a result of the conventional preview-mode image capture. Note that, in the interlace scanning, combining pieces of captured image data obtained by nine image captures is equivalent to setting the same number of points as the number of points set in the conventional measurement-mode image capture, and an image as fine as an image displayed as a result of the conventional measurement-mode image capture can be displayed.

Specifically, the position of the scan origin shifts as follows, for example. Here, AB represents a point interval on the B line, and ΔV represents a point interval on the V line.

The coordinates of the origin in the first image capture are (0, 0).

The coordinates of the origin in the second image capture are (ΔB*⅓, 0).

The coordinates of the origin in the third image capture are (ΔB*⅔, 0).

The coordinates of the origin in the fourth image capture are (0, ΔV*⅓).

The coordinates of the origin in the fifth image capture are (ΔB*⅓, ΔV*⅓).

The coordinates of the origin in the sixth image capture are (ΔB*⅔, ΔV*⅓).

The coordinates of the origin in the seventh image capture are (0, ΔV*⅔).

The coordinates of the origin in the eighth image capture are (ΔB*⅓, ΔV*⅔).

The coordinates of the origin in the ninth image capture are (ΔB*⅔, ΔV*⅔).

The coordinates of the origin in the 10th image capture are the same as the coordinates of the origin in the 1st image capture.

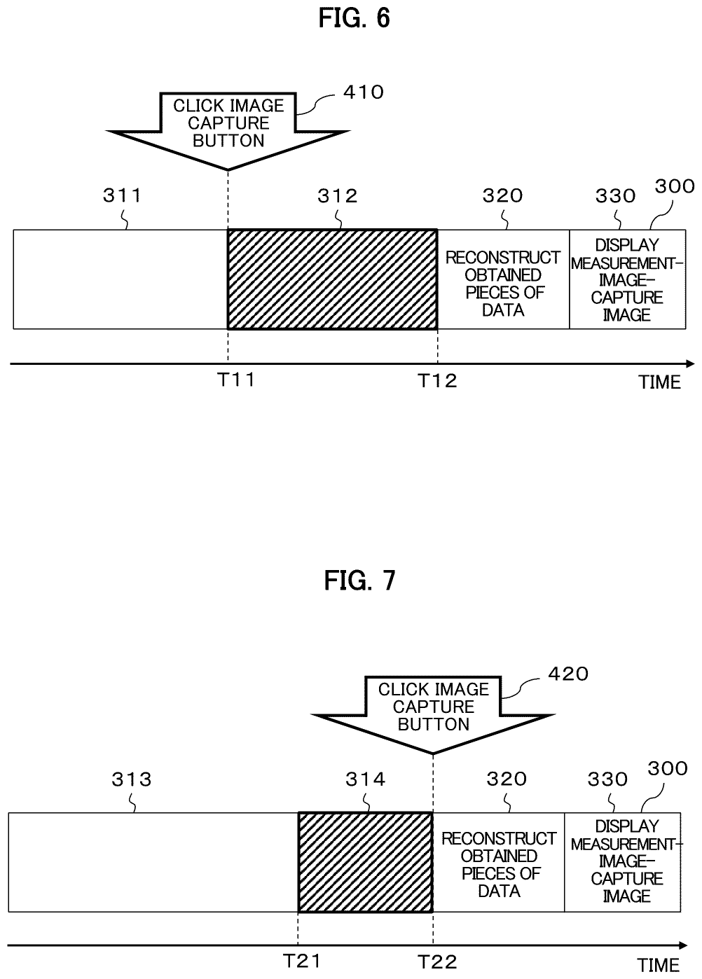

Next, a flow of an image capture process by the OCT apparatus until a measurement-image-capture image is displayed will be described with reference to FIGS. 6 and 7 (see FIG. 2 as appropriate). In FIGS. 6 and 7 and FIG. 10 to be mentioned later, the horizontal axis is a time axis, and reference sign 300 schematically shows a flow of processing when the OCT apparatus captures and displays an image of a subject. Also, the hatching shown in FIGS. 6 and 7 and FIG. 10 to be mentioned later represents obtaining of pieces of image data to be utilized in reconstruction. Note that the lengths of the processes on the time axis in each drawing may be exaggerated for clear explanation.

First of all, operation of the conventional OCT apparatus will be described as Comparative Example. When its main power is turned on, the conventional OCT apparatus performs scanning at large intervals to obtain a low-resolution image (preview-mode image capture: step 311). Looking at the preview-image-capture image (grainy OCT image) displayed on the display device, the operator positions the probe which the operator is holding to a desired spot. After completing the positioning, the operator clicks the image capture button at a time T11, for example (step 410). In response to this, the conventional OCT apparatus performs scanning at finer intervals to obtain a high-resolution image (measurement-mode image capture: step 312). During the measurement-mode image capture, the operator brings the nozzle tip of the probe of the OCT apparatus into contact with a tooth whereas the patient tries not to move for several seconds until the measurement image capture ends. The time required for the measurement image capture is set as appropriate. Here, that required time is 6 seconds, for example. Upon reaching a time T12 which is, for example, 6 seconds after the time T11, the conventional OCT apparatus uses all pieces of data obtained from the time T11 to the time T12 to reconstruct a high-resolution volume image (step 320). The conventional OCT apparatus then displays the high-resolution measurement-image-capture image (step 330).

Next, operation of the OCT apparatus 1 will be described as Example 1. When its main power is turned on, the OCT apparatus 1 performs scanning at large intervals to obtain a low-resolution image (preview-image-capture image data) (steps 313 and 314). These steps 313 and 314 correspond to the preview-mode image capture (step 311 in FIG. 6) but differ from step 311 in that the OCT apparatus 1 sequentially stores pieces of preview-image-capture image data in the memory 120. Here, the memory 120 stores at least pieces of preview-image-capture image data obtained by the last nine or more image captures and, when the storage capacity is exceeded, erases the images in chronological order. Looking at the preview-image-capture image displayed on the display device, the operator positions the probe which the operator is holding to a desired spot. After completing the positioning, the operator brings the nozzle tip of the probe into contact with a tooth whereas the patient tries not to move. Then, when determining that the same period of time as the time required in the conventional measurement image capture (e.g., 6 seconds) has elapsed, the operator clicks the image capture button at a time T22, for example (step 420). Note that a time T21 is a time preceding the time T22 by the period of time required to obtain pieces of preview-image-capture image data by a plurality of (e.g., nine) image captures. The OCT apparatus 1 then uses the pieces of preview-image-capture image data obtained by a plurality of image captures and stored in the memory 120 up to the time T22 to reconstruct a high-resolution volume image (step 320). Thereafter, the display device 54 of the OCT apparatus 1 displays the high-resolution measurement-image-capture image (step 330).

According to Example 1, it is possible to capture an image as detailed as one by the conventional measurement-mode image capture by devising the preview-mode image capture. Moreover, according to Example 1, it is possible to perform image capture without distinguishing preview-mode image capture and measurement-mode image capture as in the conventional art.

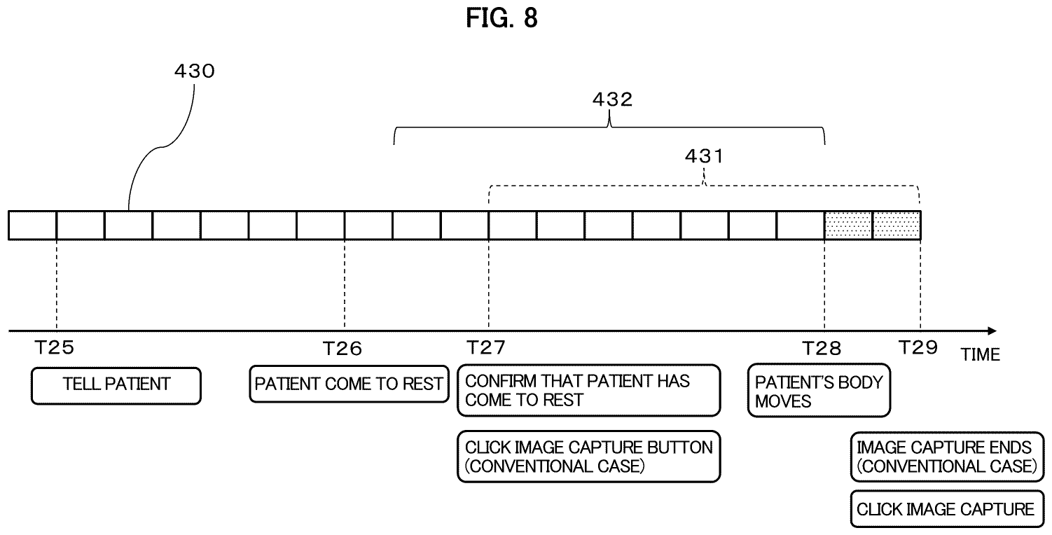

Next, an example of an advantageous effect of the OCT apparatus in Example superior to that of the conventional OCT apparatus representing Comparative Example will be described with reference to FIG. 8. The horizontal axis in FIG. 8 is a time axis representing times. The first lines under the times on this time axis describe examples of actions of the operator and the patient in association with the times. The second lines under some times on the time axis describe examples of operations on the conventional OCT apparatus in association with the times. The third line under a time on the time axis describes an example of an operation on the OCT apparatus in Example in association with the time. Also, a group of pieces of preview image data 430 shown above the time axis represents pieces of preview image data which the OCT apparatus in Example obtained by a plurality of image captures and stored in the memory within a predetermined period of time. Here, each individual rectangle linked to one another in the direction of time schematically represents a piece of preview image data.

Examples of actions of the operator and the patient will be described first.

Δt a time T25, the operator positions the probe at a desired spot and brings the nozzle tip of the probe into contact with a tooth, and at the same time says “Please do not move your body and stay still now.”, for example, to the patient. Several seconds elapses while the operator says this single phrase. At a time T26 after the operator says this single phrase, the patient's body comes to rest. Thereafter, at a time T27, the operator confirms that the patient's body has come to rest. Several seconds elapses while the operator confirms that the patient's body has come to rest.

In the case of using the conventional OCT apparatus, the operator clicks the image capture button at the time T27 after confirming that the patient's body has come to rest. As a result, measurement-mode image capture starts.

Then, at a time T29 after a predetermined period of time set in the conventional OCT apparatus in advance, the conventional OCT apparatus finishes obtaining pieces of image data to be utilized in reconstruction, and the conventional image capture ends. Then, at a time T29 which is the time when the image capture ends, the operator says “The image capture is finished.” to the patient. If the patient's body does not move until the image capture end time, the conventional OCT apparatus reconstructs the pieces of image data obtained by the one measurement-mode image capture (one volume) performed from the time T27 to the time T29 and obtains a good image appropriate for storage. If, however, the patient's body moves in the period from a time T28, which is before the end of the conventional image capture, to the time T29, the conventional OCT apparatus obtains an image not appropriate for storage. In this case, it is necessary to redo the image capture.

In Example, on the other hand, the measurement-mode image capture is not needed, and therefore the operator does not click the image capture button at the time T27 after confirming that the patient's body comes to rest.

Then, at the time T29 which is the time when the image capture ends, the operator clicks the image capture button and says “The image capture is finished.” to the patient. If the patient's body does not move until this image capture end time, the OCT apparatus 1 generates measurement-image-capture image data by using, for example, the nine pieces of preview image data included in the section of the group of pieces of preview image data 430 denoted by reference sign 431, i.e., the section from the time T27 to the time T29. As a result, a good image appropriate for storage is obtained. Also, even if the patient's body moves in the period from the time T28 to the time T29, the OCT apparatus 1 generates measurement-image-capture image data by using the nine pieces of preview image data included in the section of the group of pieces of preview image data 430 denoted by reference sign 432, which precedes the section denoted by reference sign 431. As a result, a good image appropriate for storage is obtained. In this case, it is not necessary to redo the image capture. Meanwhile, the dots shown in some rectangles in FIG. 8 schematically show that the pieces of preview image data are affected by the movement of the patient's body or the like.

In short, comparing Comparative Example and Example under the same conditions, Example is effective in reducing the number of times to redo the image capture in the case where the patient's body moves or the image capture probe is moved. Moreover, as compared to Comparative Example, Example is effective in reducing the number of times to redo the image capture also in the case where blood, saliva, or the like oozes over the patient's tooth in the period from the time T28 to the time T29.

### Second Embodiment

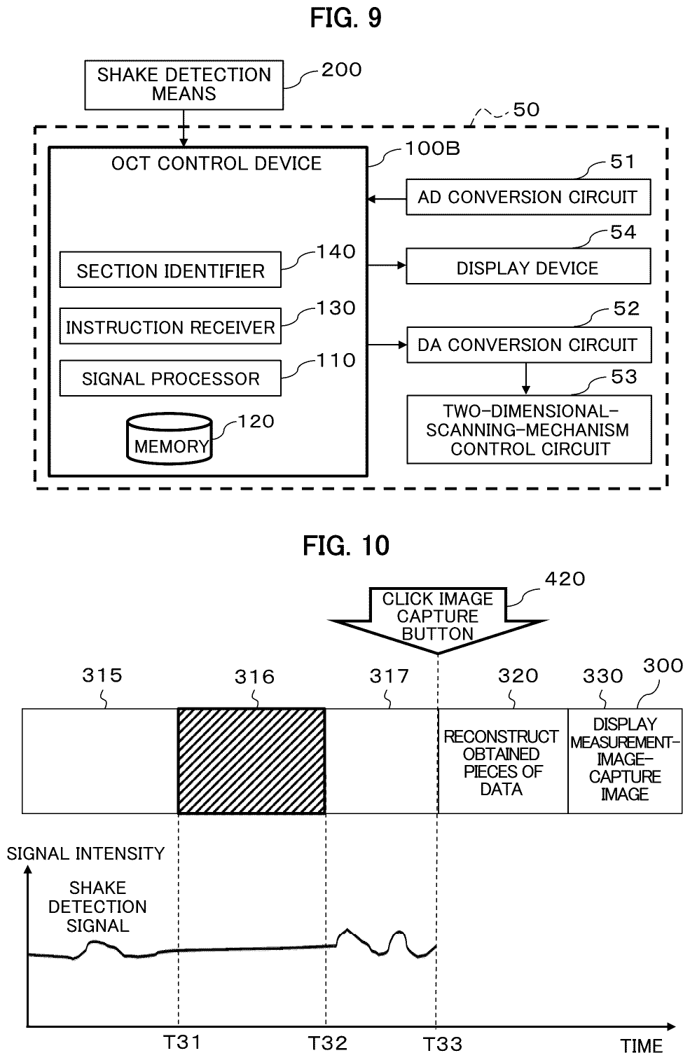

Next, an OCT apparatus according to a second embodiment will be described focusing on its OCT control device with reference to FIG. 9. Note that a diagram of the entire OCT apparatus according to the second embodiment would be the same as FIG. 1, and is therefore omitted. Moreover, the same components as those of the OCT control device 100 in FIG. 2 are denoted by the same reference signs, and description thereof is omitted. The OCT apparatus according to the second embodiment includes a shake detection means 200. The shake detection means 200 generates a shake detection signal indicating a temporal change in shaking of the probe 30 held by the operator. In the present embodiment, the shake detection means 200 is a sensor that is incorporated in the probe 30 and senses positional changes. Here, the sensor that senses positional changes is, for example, a sensor such as an acceleration sensor, a gyro sensor, a displacement sensor, or a vibration sensor that, in response to sensing vibration, converts it into an electrical signal.

An OCT control device 100B according to the second embodiment includes the signal processor 110, the memory 120, the instruction receiver 130, and a section identifier 140.

The section identifier 140 identifies a time section in which the intensity of the waveform of the shake detection signal does not continuously exceed a preset threshold value. In response to receiving the shake detection signal generated by the shake detection means 200, the section identifier 140 automatically sets a time section in which shake is small based on the waveform of the shake detection signal. The section identifier 140 stores the shake detection signal in the memory 120 and outputs the time section identified from the shake detection signal to the signal processor 110. The memory 120 stores the shake detection signal in synchronization with the pieces of preview image data.

The signal processor 110 generates measurement-image-capture image data by using the pieces of preview image data obtained in the time section identified by the section identifier 140 among the pieces of preview image data obtained by a plurality of image captures and stored in the memory 120 within a predetermined period of time up to the point when the instruction receiver 130 received an instruction.

Next, a flow of an image capture process by the OCT apparatus according to the second embodiment until a measurement-image-capture image is displayed will be described with reference to FIG. 10. Note that elements similar to those in FIGS. 6 and 7 are denoted by the same reference signs, and similar description is omitted. In FIG. 10, the horizontal axis represents a time axis, and the vertical axis represents signal intensity. Moreover, FIG. 10 shows a timing chart of processing performed by the OCT apparatus at the time of capturing and displaying an image of a subject, as well as the shake detection signal.

When its main power is turned on, the OCT apparatus 1 according to the second embodiment performs scanning at large intervals to obtain a low-resolution image (preview-image-capture image data) (steps 315, 316, and 317). The OCT apparatus 1 differs from the OCT apparatus according to the first embodiment in that the former at this time sequentially stores the signal intensity of the shake detection signal in the memory 120 in association with the preview-image-capture image data. After positioning the probe while studying the preview-image-capture image, the operator brings the nozzle tip of the probe into contact with a tooth. Then, when determining that a predetermined period of time (e.g., 6 seconds) has elapsed, the operator clicks the image capture button at a time T33, for example (step 420).

Note that, in FIG. 10, the amplitude of the signal intensity of the shake detection signal is higher than a predetermined threshold value in the section before a time T31 and the section from a time T32 to the time T33. For this reason, the pieces of data obtained in steps 315 and 317 are pieces of data that should not be utilized in the image reconstruction.

Based on the shake detection signal, the OCT apparatus 1 according to the second embodiment identifies, for example, the section from the time T31 to the time T32 as a section in which shake is small. In this case, the OCT apparatus 1 reconstructs a high-resolution volume image by using the pieces of preview-image-capture image data obtained by a plurality of (e.g., nine) image captures and stored in the memory 120 in step 316 in association with the shake detection signal detected in the period from the time T31 to the time T32 (step 320). Thereafter, the display device 54 of the OCT apparatus 1 displays the high-resolution measurement-image-capture image (step 330).

According to the present embodiment, it is possible to obtain a good, less blurry image. Incidentally, various modifications can be made to the second embodiment. In the above example, the OCT apparatus 1 according to the second embodiment generates measurement-image-capture image data generally by reconstructing a preset number (nine) of pieces of preview image data in a section in which shake is small. Here, there is no guarantee that the preset number (nine) of pieces of preview image data will always be obtained in a section where shake is small. Thus, if the number of pieces of preview image data in a section where shake is small is less than the preset number (nine), all pieces of data in that identified section can be used in the reconstruction (first modification). In this case, the image quality is not as good as when the preset number (nine) of pieces of preview image data are used. Nonetheless, a less blurry image can still be obtained.

Also, if the number of pieces of preview image data in a section where shake is small is more than the preset number (nine), all pieces of data in that identified section can be used in the reconstruction (second modification). By reconstructing a larger number of pieces of data than in the normal image capture as above, it is possible to obtain a less blurry image with higher image quality.

### Third Embodiment

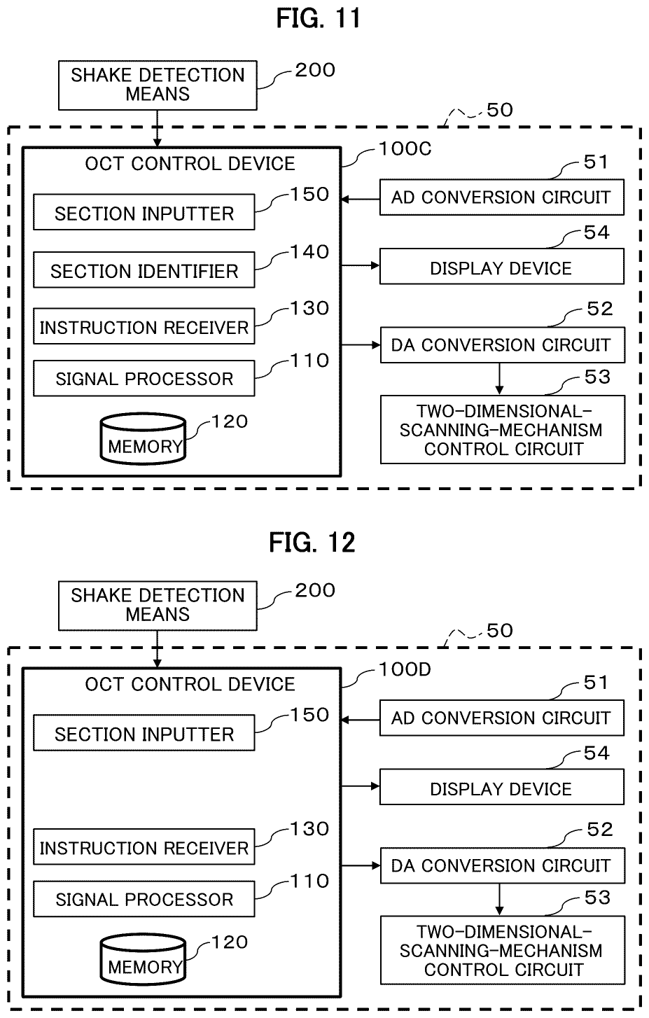

Next, an OCT apparatus according to a third embodiment will be described focusing on its OCT control device with reference to FIG. 11. An OCT control device 100C of the OCT apparatus according to the third embodiment includes the signal processor 110, the memory 120, the instruction receiver 130, the automatic-type section identifier 140, and a manual-type section inputter 150. The OCT apparatus according to the third embodiment differs from the OCT apparatus according to the second embodiment in that the former includes the section inputter 150, and the display device 54 displays the waveform of the shake detection signal. That is, the OCT control device 100C according to the third embodiment is also capable of displaying a measurement-image-capture image representing a section automatically set by the section identifier 140 in which shake is small.

The section inputter 150 displays the waveform of the shake detection signal on the display device 54 such that the operator can select a continuous time section in the waveform of the shake detection signal, and inputs a time section selected by the operator. In response to receiving a time section selected by the operator from the section inputter 150, the signal processor 110 generates measurement-image-capture image data by using the pieces of preview image data obtained in that time section.

According to the present embodiment, the operator can check the waveform of the shake detection signal displayed on the display device 54 and select a section him- or herself in which shake is small to thereby correct the section automatically set by the section identifier 140.

### Fourth Embodiment

Next, an OCT apparatus according to a fourth embodiment will be described focusing on its OCT control device with reference to FIG. 12. An OCT control device 100D of the OCT apparatus according to the fourth embodiment includes the signal processor 110, the memory 120, the instruction receiver 130, and the section inputter 150. The OCT control device 100D according to the fourth embodiment differs from the OCT control device 100C according to the third embodiment in that a section in which shake is small is set manually, instead of being set automatically.

In response to receiving a time section selected by the operator from the section inputter 150, the signal processor 110 generates measurement-image-capture image data by using the pieces of preview image data obtained in that time section among the pieces of preview image data obtained by a plurality of image captures and stored in the memory 120 within a predetermined period of time up to the point when the instruction receiver 130 received an instruction.

According to the present embodiment, the operator can check the waveform of the shake detection signal displayed on the display device 54 and select and set a section him- or herself in which shake is small.

### Fifth Embodiment

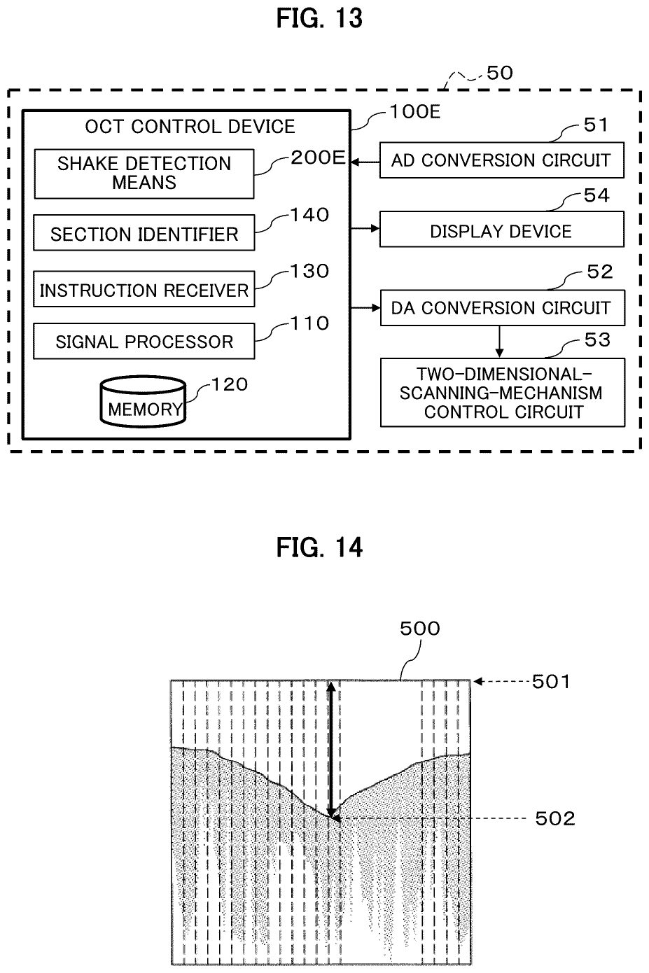

Next, an OCT apparatus according to a fifth embodiment will be described focusing on its OCT control device with reference to FIG. 13 (see FIG. 9 as appropriate). In the second embodiment described earlier, the shake detection means 200 in FIG. 9 is a sensor that is incorporated in the probe 30 and senses positional changes. In the present embodiment, an OCT control device 100E includes a shake detection means 200E. Specifically, the shake detection means 200E is not an acceleration sensor or a gyro sensor, and analyzes an OCT image by image processing, generates a shake detection signal indicating a temporal change in shaking of the probe 30 held by the operator based on the result of the analysis, and outputs the shake detection signal to the section identifier 140.

The shake detection means 200E calculates the distance from the upper edge of an A-sectional image to the upper surface of the subject in that sectional image, and generates a shake detection signal based on the temporal change in the calculated distance. Specifically, in the case of the preview-mode image capture conditions mentioned earlier (A line: 1024 points, B line: 134 points, V line: 134 points), the median of each of the coordinate values on the B line (B) and the coordinate values on the V line (V) is 67. The shake detection means 200E can then use the A-sectional image at the center coordinates (B, V)=(67, 67), which is obtained by one preview-mode image capture (one volume), for example. FIG. 14 is a diagram showing an example of the A-sectional image at the center coordinates (B, V)=(67, 67) in one volume. In this case, the shake detection means 200E can use the distance from the upper edge of an A-sectional image 500 shown in FIG. 14 (the position indicated by reference sign 501) to the upper surface of the subject (the position indicated by reference sign 502) (the distance indicated by the double-headed arrow in FIG. 14). Also, instead of the distance at the single point at the center coordinates (B, V)=(67, 67) in the one volume, the distance at an adjacent coordinate value on the B line around that point can be used, or an average value derived by averaging the distances at such adjacent coordinate values on the B line can be used. This case is effective in making the measurement-image-capture image data less vulnerable to noise and the like, and is therefore preferable.

### Sixth Embodiment

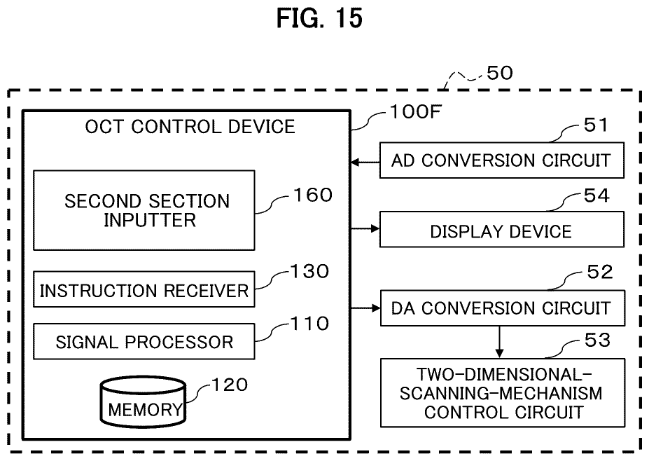

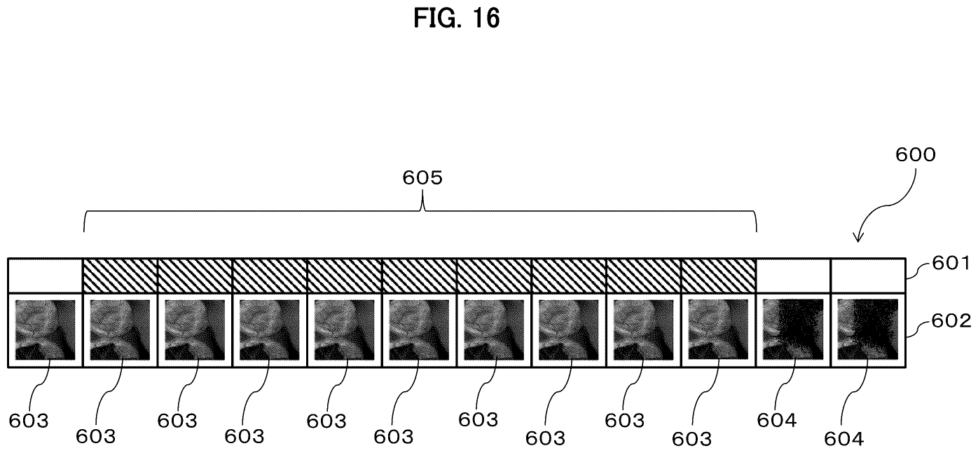

Next, as a sixth embodiment, an OCT apparatus which obtains a good image appropriate for storage by using preview images in a section in which blood or the like is not oozing over a patient's tooth will be described focusing on its OCT control device with reference to FIG. 15 (see FIG. 2 as appropriate). Note that a diagram of the entire OCT apparatus according to the sixth embodiment would be the same as FIG. 1, and is therefore omitted. Moreover, the same components as those of the OCT control device 100 in FIG. 2 are denoted by the same reference signs, and description thereof is omitted.

An OCT control device 100F according to the sixth embodiment includes the signal processor 110, the memory 120, the instruction receiver 130, and a manual-type second section inputter 160.

In the present embodiment, the signal processor 110 generates an en-face image per piece of preview image data (volume). Here, an en-face image is a two-dimensional image obtained by combining pieces of subject-hight-direction data in a piece of preview image data. The second section inputter 160 displays a plurality of en-face images on the display device 54 such that the operator can select a time section in which a series of en-face images are arranged, and inputs a time section selected by the operator. In response to receiving a time section selected by the operator from the second section inputter 160, the signal processor 110 generates measurement-image-capture image data by using the pieces of preview image data obtained in that time section among the pieces of preview image data obtained by a plurality of image captures and stored in the memory 120 within a predetermined period of time up to the point when the instruction receiver 130 received an instruction.

Here, the signal processor 110 is capable of synchronizing the timing of generating en-face images with the operation of the second section inputter 160, for example. In this case, when the second section inputter 160 displays en-face images on the display device 54, the signal processor 110 generates those en-face images from pieces of preview image data (volume data) stored in the memory 120.

Each time the signal processor 110 obtains a piece of preview image data, the signal processor 110 can generate an en-face image from the piece of preview image data and store the en-face image in the memory 120 in association with the piece of preview image data. In this manner, the second section inputter 160 can quickly display an en-face image on the display device 54 by simply reading that en-face image out of the memory 120.

Next, a specific example in which the second section inputter 160 displays a plurality of en-face images on the display device 54 will be described with reference to FIG. 16. FIG. 16 is a display example of a screen on the display device 54 of the OCT apparatus according to the sixth embodiment. On the screen on the display device 54, for example, a section input screen 600 is displayed as shown in FIG. 16. The section input screen 600 includes a section selection region 601 and an en-face image display region 602 arranged under this section selection region 601. In the section selection region 601, a plurality of rectangles are displayed which are arranged in an array and correspond to a plurality of time sections. Here, each individual rectangle linked to one another in the screen width direction (horizontal direction) schematically represent a time section for a single preview-mode image capture. Also, the time section represented by a rectangle arranged on the right is a time section after the time section represented by a rectangle arranged on the left.

In the en-face image display region 602, for each time section, an en-face image 603 or 604 is displayed which is based on the piece of preview image data obtained in that section. Each en-face image is a two-dimensional image obtained by combining pieces of hight-direction data in the subject's three-dimensional image. For this reason, if blood or the like oozes over the subject during the image capture, the blood or the like oozing over the subject is depicted in the en-face image, as a matter of course. The oozed blood or the like is displayed like a stain. Thus, the operator can find the oozing of the blood or the like by visually checking the en-face image. Here, each en-face image 603 represents a normal en-face image. Each en-face image 604 represents an en-face image with oozed blood.

In the example shown in FIG. 16, nine sections across which normal en-face images 603 without a stain are arranged in series are selected, as indicated by reference sign 605 on the section selection region 601. Here, the selected sections are indicated by hatching the rectangles in the section selection region 601. Each rectangle in the section selection region 601 can be configured as a checkbox so that the operator can check it with a mouse or the like.

Since the en-face images are displayed in an array in the en-face image display region 602, the size of each image is small, which may make it difficult to discriminate a stain in the image. To address this, each en-face image can be thumbnailed. In this case, the operator can click a thumbnailed en-face image with a mouse or the like to display an enlarged version of the clicked en-face image. In this way, it is easier for the operator to distinguish a normal en-face image 603 without a stain and an en-face image 604 with a stain.

Also, if shake occurs during an image capture (while a preview image is obtained), the en-face image gets distorted. Thus, it is possible to check blur by visually checking the en-face image. This enables the operator to select a section in which normal en-face images without blur or a stain are arranged in series.

Although not shown in drawings, the sixth embodiment can be modified to include a shake detection means as follows.

(Modification 1) The OCT apparatus according to the sixth embodiment can further include the shake detection means 200 and the section identifier 140 shown in FIG. 9.

(Modification 2) The OCT apparatus according to the sixth embodiment can further include the shake detection means 200, the section identifier 140, and the section inputter 150 shown in FIG. 11.

(Modification 3) The OCT apparatus according to the sixth embodiment can further include the shake detection means 200 and the section inputter 150 shown in FIG. 12.

(Modification 4) The OCT apparatus according to the sixth embodiment can further include the shake detection means 200E and the section identifier 140 shown in FIG. 13.

(Modification 5) The shake detection meanss 200 in the OCT apparatuses according to Modifications 1 to 3 can be replaced with the shake detection means 200E.

The present invention can be implemented with a program that causes hardware resources such as a CPU, a memory, and a hard disk drive included in a computer to operate in a coordinated manner as the OCT control device 100 (OCT apparatus controlling program). This program can be distributed through a communication line or written to a recording medium, such as a CD-ROM or a flash memory, and distributed.

Note that, while embodiments of the present invention have been described in detail, the present invention is not limited to the embodiments described above, and encompasses design changes and the like without departing from the gist of the present invention. For example, in the above embodiments, a mechanical method is employed as the method for shifting the scan origin. Alternatively, a software method can be employed. In that case, the signal processor 110 can take in the mirror coordinates at the two-dimensional scanning mechanism 33 and, each time the signal processor 110 obtains a piece of preview image data, shift the minor coordinates from which to start the obtaining of data of an A-sectional image to thereby shift the scan origin (origin of two-dimensional scanning).

A case where galvanometer mirrors are employed as the two-dimensional scanning mechanism 33 has been described. However, the present invention is not limited to this, and can employ two-dimensional MEMS mirrors. A two-dimensional MEMS minor has, for example, elements formed in a three-layer structure including: a silicon layer where movable structures are formed such as a mirror that totally reflects light and a planar coil for electromagnetic driving that generates an electromagnetic force; a ceramic base; and a permanent magnet, and can be controlled to statically and dynamically tilted in an X-axis direction and a Y-axis direction in proportion to the level of a current applied to the coil.

### REFERENCE SIGNS LIST

1 OCT apparatus
10 optical unit
11 light source
12 coupler
13 sample arm
14 circulator
polarization controller
16 coupler
17 reference arm
18 circulator
19 collimator lens
20 condensing lens
21 reference minor
22 polarization controller
23 detector
30 probe
31 shutter
32 collimator lens
33 two-dimensional scanning mechanism
34 condensing lens
50 control unit
51 AD conversion circuit
52 DA conversion circuit
53 two-dimensional-scanning-mechanism control circuit
54 display device
100, 100B, 100C, 100D, 100E, 100F OCT control device
110 signal processor
120 memory
130 instruction receiver
140 section identifier
150 section inputter
160 second section inputter
200, 200E shake detection means
S subject

1. An OCT apparatus for measuring internal information of a subject by optical coherence by irradiating the subject with a laser beam from a probe including a two-dimensional scanning mechanism that two-dimensionally scans a laser beam, the OCT apparatus comprising:
a signal processor that obtains preview image data corresponding to a volume image with predetermined graininess by a single image capture, the volume image being sectional images of cross sections along an optical axis of a laser beam stacked in a direction perpendicular to the cross sections by measuring subject information in a direction along the optical axis of the laser beam at a preset point interval for two-dimensional scanning;
a memory that stores pieces of the preview image data obtained by a plurality of image captures; and
an instruction receiver that receives an instruction to generate measurement-image-capture image data corresponding to a volume image of the subject with finer graininess than the predetermined graininess, wherein
the signal processor obtains pieces of the preview image data by a plurality of image captures with an origin of the two-dimensional scanning shifted by a small interval smaller than the point interval so as to divide the point interval each time the signal processor obtains a piece of the preview image data, and generates the measurement-image-capture image data by reconstructing pieces of the preview image data obtained by a plurality of image captures and stored in the memory up to a point when the instruction was received.

2. The OCT apparatus according to claim 1, further comprising:
a shake detection means that generates a shake detection signal indicating a temporal change in shaking of the probe held by an operator; and
a section identifier that identifies a time section in which intensity of a waveform of the shake detection signal does not continuously exceed a preset threshold value, wherein
the memory stores the shake detection signal in synchronization with the pieces of preview image data, and
the signal processor generates the measurement-image-capture image data by using pieces of the preview image data obtained in the identified time section among pieces of the preview image data obtained by a plurality of image captures and stored in the memory within a predetermined period of time up to the point when the instruction was received.

3. The OCT apparatus according to claim 2, further comprising
a section inputter that displays the waveform of the shake detection signal on a display device such that the operator can select a continuous time section in the waveform of the shake detection signal, and inputs a time section selected by the operator, wherein
in response to receiving a time section selected by the operator from the section inputter, the signal processor generates the measurement-image-capture image data by using pieces of the preview image data obtained in the time section.

4. The OCT apparatus according to claim 1, further comprising:
a shake detection means that generates a shake detection signal indicating a temporal change in shaking of the probe held by an operator; and
a section inputter that displays the waveform of the shake detection signal on a display device such that the operator can select a continuous time section in the waveform of the shake detection signal, and inputs a time section selected by the operator, wherein
the memory stores the shake detection signal in synchronization with the pieces of preview image data, and
in response to receiving a time section selected by the operator from the section inputter, the signal processor generates the measurement-image-capture image data by using pieces of the preview image data obtained in the time section among pieces of the preview image data obtained by a plurality of image captures and stored in the memory within a predetermined period of time up to the point when the instruction was received.

5. The OCT apparatus according to claim 2, wherein the shake detection means is a sensor that is incorporated in the probe and senses a positional change.

6. The OCT apparatus according to claim 2, wherein the shake detection means calculates a distance from an upper edge of at least one of the sectional images to an upper surface of the subject in the at least one sectional image, and generates the shake detection signal based on a temporal change in the calculated distance.

7. The OCT apparatus according to claim 1, wherein
the signal processor generates an en-face image for each piece of the preview image data, the en-face image being a two-dimensional image obtained by combining pieces of subject-hight-direction data in the piece of preview image data,
the OCT apparatus further comprises a second section inputter that displays a plurality of the en-face images on a display device such that an operator can select a time section in which a plurality of the en-face images are arranged in series, and inputs a time section selected by the operator, and
in response to receiving a time section selected by the operator from the second section inputter, the signal processor generates the measurement-image-capture image data by using pieces of the preview image data obtained in the time section among pieces of the preview image data obtained by a plurality of image captures and stored in the memory within a predetermined period of time up to the point when the instruction was received.

8. The OCT apparatus according to claim 7, wherein when the second section inputter displays the en-face images on the display device, the signal processor generates the en-face images from the pieces of preview image data stored in the memory.

9. The OCT apparatus according to claim 7, wherein each time the signal processor obtains a piece of the preview image data, the signal processor generates the en-face image from the obtained piece of preview image data, and stores the generated en-face image in the memory in association with the piece of preview image data.

10. The OCT apparatus according to claim 1, wherein the memory sequentially stores pieces of the preview image data obtained by a plurality of image captures and, when a storage capacity is exceeded, sequentially erases the pieces of preview image data in chronological order.

11. The OCT apparatus according to claim 1, wherein each time the signal processor obtains a piece of the preview image data, the signal processor shifts the origin of the two-dimensional scanning by outputting a signal for shifting an origin of a scanning start position at the two-dimensional scanning mechanism to a two-dimensional-scanning-mechanism control circuit that controls the two-dimensional scanning mechanism.

12. The OCT apparatus according to claim 1, wherein the signal processor takes in a mirror coordinate at the two-dimensional scanning mechanism and, each time the signal processor obtains a piece of the preview image data, shifts the mirror coordinate from which to start obtaining of data of the sectional images to thereby shift the origin of the two-dimensional scanning.

13. A method for controlling an OCT apparatus for measuring internal information of a subject by optical coherence by irradiating the subject with a laser beam from a probe including a two-dimensional scanning mechanism that two-dimensionally scans a laser beam, the method comprising:
a step of obtaining preview image data corresponding to a volume image with predetermined graininess by a single image capture, the volume image being sectional images of cross sections along an optical axis of a laser beam stacked in a direction perpendicular to the cross sections by measuring subject information in a direction along the optical axis of the laser beam at a preset point interval for two-dimensional scanning;
a step of shifting an origin of the two-dimensional scanning by a small interval smaller than the point interval so as to divide the point interval each time a piece of the preview image data is obtained;
a step of storing pieces of the preview image data obtained by a plurality of image captures in a memory;
a step of receiving an instruction to generate measurement-image-capture image data corresponding to a volume image of the subject with finer graininess than the predetermined graininess; and
a step of generating the measurement-image-capture image data by reconstructing pieces of the preview image data obtained by a plurality of image captures and stored in the memory up to a point when the instruction was received.

14. A non-transitory computer-readable storage medium that stores therein an OCT apparatus controlling program for causing a computer to function as a control device of the OCT apparatus according to claim 1.
